(19) United States
(12) Patent Application Publication
(10) Pub. No.: US 2023/0182385 A1
(43) Pub. Date: Jun. 15, 2023

(54) ADDITIVE MANUFACTURING COMPONENTS AND METHODS

(71) Applicant: Meta Additive Ltd., Stoke-On-Trent (GB)

(72) Inventors: Kate BLACK, Liverpool (GB); Paul CHALKER, Liverpool (GB); Christopher J SUTCLIFFE, Liverpool (GB)

(21) Appl. No.: 17/921,995

(22) PCT Filed: Apr. 28, 2021
(86) PCT No.: PCT/GB2021/051023

§ 371 Date: Oct. 28, 2022

(30) Foreign Application Priority Data
May 1, 2020 (GB) 2006473.9

(51) Int. Cl. B29C 64/209; B22F 10/16; B29C 64/112; B29C 64/165; B22F 10/14; B22F 10/50; B22F 10/85; B22F 10/64

(52) U.S. Cl. CPC B29C 64/209; B22F 10/16; B29C 64/112; B29C 64/165; B22F 10/14; B22F 10/50; B22F 10/85; B22F 10/64; B33Y 10/00

(57) ABSTRACT

A method of 3D printing in which a 3D product is built up layer by layer by jetting from print heads includes forming part of a 3D product by a functional binder jetting process; jetting one or more material in a 2D pattern to form a structure on said part; completing the formation of the 3D product by continuing the functional binder jetting process, so that said structure becomes embedded in said product. Functional binder jetting may include: providing a layer of a powder bed; jetting a functional binder onto selected parts of said layer, wherein said functional binder infiltrates into pores in the powder bed and locally fuses particles of the powder bed in situ; sequentially repeating applying a layer of powder on top and selectively jetting functional binder, multiple times, to provide a powder bed bonded at selected locations by printed functional binder.

### CROSS-REFERENCE TO RELATED APPLICATIONS

This application is a U.S. National Stage Application of International Application No. PCT/GB2021/051023, filed on Apr. 28, 2021, and claims priority under 35 U.S.C. § 119(a) and 35 U.S.C. § 365(b) from GB Patent Application No. 2006473.9, filed on May 1, 2020; the disclosures of which are incorporated herein by reference.

### FIELD

Various embodiments of the present disclosure relate to additive manufacturing, also known as 3D printing, and in particular to binder jetting, components used in binder jetting, and resultant products.

### BACKGROUND

Additive manufacturing, commonly referred to as 3D printing, is a term which encompasses several categories of processes by which 3D objects are formed or “printed”. The 3D objects are generally built up layer by layer, and the processes differ in the way that the layers are formed and in what they are made from.

Some processes entail polymerizing or curing liquid material. For example, in vat photopolymerization, a platform is lowered into a vat of liquid polymerizable material (e.g. epoxy acrylate resin) so that it is slightly below the surface. Laser radiation is used to polymerize and harden selective parts of the layer above the platform. The platform is then lowered slightly so that a new liquid layer is at the surface (this may be made uniform by using a levelling or coating blade) and the polymerization process is repeated. This procedure of lowering, coating and polymerizing is repeated layer by layer until the desired three-dimensional structure has been formed. The platform may then be raised and the product removed and processed further. Post-processing typically involves the removal of support structures (which may be formed during the polymerization steps) and any other residual material, and then high temperature curing following by finishing, e.g. sanding of the product. Some other processes entail forming each layer of a 3D structure by extruding a plastic or polymer material (or, less commonly, other material). This is known as extrusion deposition or fused deposition modelling (FDM). Material, e.g. a polylactic acid resin, is fed to an extruder where it is heated and extruded through a nozzle which moves in X and Y directions. The selectively deposited material solidifies on cooling. As with vat polymerization methods, the structure usually rests on a build platform which typically moves downwards between the deposition of each layer, and support structures are typically required, particularly for overhanging parts of structures. Such extrusion methods are amongst the most common 3D printing processes and used widely in consumer 3D printers. Another category of additive manufacturing is material jetting which is similar to extrusion deposition in that material is deposited via a nozzle which moves in X and Y directions. Instead of being extruded, the material is jetted onto a platform. The material (e.g. wax or polymer) is applied as droplets using a print head, similar to conventional two-dimensional inkjet printing. The droplets solidify and then successive layers are applied. Once the structure is formed it may be subjected to curing and post-processing. As with other methods discussed above, support structures may be incorporated during the procedure and then removed during post processing.

Powder bed fusion (PBF) methods entail the selective binding of granular materials. This can be done by melting and fusing together part of the powder or particles of a layer of material, then lowering the bed, adding a further layer of powder and repeating the melting and fusing process. The unfused powder around the fused material provides support so unlike some methods discussed above it may not be necessary to use support structures. Such methods include direct metal laser sintering (DMLS), electron beam melting (EBM), selective heat sintering (SHS), selective laser melting (SLM) and selective laser sintering (SLS). In view of the types of materials which are compatible with such processes (including metals and polymers), functional high strength materials can be manufactured.

Binder jetting methods are similar to powder bed fusion methods in that they use layers of powder or particulate material. However, conventional binder jetting methods differ from powder bed fusion methods in that the powder is not initially fused together but instead is held together with a binder which is jetted onto the structure from a print head. The binder may be colored and the color may be imparted to the powder thereby allowing color 3D printing. Typically a binder is applied in a specific pattern to a layer of powder, and then the steps of applying a layer of powder and selectively applying binder are repeated.

In general, binder jetting entails the use of binder as a sacrificial material which is altered or removed in a post-processing step. This is because the adhesive binder typically imparts enough mechanical strength (termed “green strength”) to enable the structure to be self-supporting and maintain its shape as it is built up, and to withstand mechanical operations during manufacture, but not enough strength to be functional for the intended end use. Thus the structure is usually subsequently heated to remove the binder (de-binding process) and to fuse the build material together in a post-processing step to ensure that the product is fit for purpose which may include load-bearing or other applications.

Binder jetting is also referred to as the “drop-on” technique, “powder bed and inkjet 3D printing”, or sometimes just “3D printing”, though as summarized here there are many other different types of 3D printing. The binder used in binder jetting is generally liquid and is often referred to as “ink” in view of the inkjet application process.

One challenge with traditional binder jetting relates to porosity. The post-processing heat treatment step removes the binder and fuses the structure, but leaves significant porosity. This is partly due to the inherent packing densities which are possible with the particles of the powder bed, and partly due to the de-binding process. The de-binding process can also cause further problems, in particular shrinkage and contamination. The pores which remain can compromise mechanical properties. A further step of infiltration can be used to fill the pores, but this adds complexity and generally requires a different type of material so that the end product is generally weaker than an equivalent material made from a single material and is more difficult to recycle.

Yet further methods of 3D printing include lamination methods (wherein single sheets are formed and laminated together), and directed energy deposition (where powder is supplied to a surface and melted on deposition by e.g. a laser beam).

An Innovate UK assessment estimated the worldwide market for all additive manufacturing products and services to be worth $4.1 billion in 2014. Currently the sector has experienced a compound annual global growth rate of 35% over the last three years, driven by direct part production, which now represents 43% of the total revenue (“Shaping our National Competency in Additive Manufacturing”, 2012: https://connect.innovateuk.orq) Future growth is forecast to be about $21 billion by 2020, which is expected to be driven by the adoption of additive manufacturing by the aerospace, medical devices, automotive and creative industries (“3D Printing and Additive Manufacturing State of the Industry,” W. A. Fort Collins, Editor 2014). Additive manufacturing has become a core technology within the field of high value manufacturing. Metals are the fastest-growing segment of the additive manufacturing sector, with printer sales growing at 48% and material sales increasing by 32% (Harrop, R. G. A. J., 3D Printing of Metals 2015-2025 Pricing, properties and projections for 3D printing equipment, materials and applications, IDTechEX, 2015.). Campbell et al (Campbell L, R. I., Bourell, D. and Gibson, I., “Additive manufacturing: rapid prototyping comes of age,” Rapid Prototyping Journal, 2012, 18(4): p. 255) have noted that the industry drivers for the development of additive manufacturing technology can be differentiated as:
Automotive—the ability to deliver new products to market quickly and predictably, significantly reduces overall vehicle development costs.
Aerospace—realization of highly complex and high performance parts with integrated mechanical function, elimination of assembly features and enabling the creation of internal functionality (e.g. cooling etc.)
Medical—translation of 3D medical imaging data into customized solid medical devices, implants and prostheses.

Additive manufacturing is regarded as a disruptive technology that could be revolutionary and game changing, if barriers such as inconsistent material properties can be overcome. The present invention directly addresses this issue.

A known additive manufacturing method uses functional binders in a jetting process, including reactive metal jet fusion (RMJF) printing. This addresses some of the problems outlined above by jetting a material which not only binds selected parts of a powder bed layer together, but also becomes part of the build material: the binder is functional rather than sacrificial. The powder bed particles are fused in situ by application of the binder.

### BRIEF DESCRIPTION OF THE DRAWINGS

In the following description, characteristics and advantages of the present disclosure will become more apparent from the following detailed description of the various embodiments with reference to the attached drawings, in which:

FIG. 1 is a schematic representation of an apparatus used and components prepared according to one exemplary embodiment of a method of 3D printing in accordance with an aspect of the present disclosure; and

FIGS. 2a, 2b, and 2c are schematic representations of incomplete, part-finished and finished components prepared in accordance with an exemplary embodiment of the present invention.

### DETAILED DESCRIPTION

Various embodiments of the present disclosure relate to functional binder jetting printing which bring further improvements and advantages, and in particular to methods which are particularly effective in the preparation of parts with embedded structures.

From a first aspect, the present disclosure provides a method of 3D printing in which a 3D product is built up layer by layer by jetting from print heads, comprising:

(a) forming part of a 3D product by a functional binder jetting process;

(b) jetting one or more material in a 2D pattern to form a structure on said part; (c) completing the formation of the 3D product by continuing the functional binder jetting process, so that said structure becomes embedded in said product.

Thus, exemplary embodiments of the present disclosure invention use a functional binder jetting process to build up the bulk of the 3D product which surrounds an embedded structure.

A “functional binder jetting process”, as described in WO 2019/025801, is a process which differs from a more conventional binder jetting process in that a functional binder is used to bind powder particles in a powder bed. “Functional” in this context means that the binder not only binds together build material but also becomes part of the build material. This functional binder jetting process can entail:

(i) providing a layer of a powder bed;

(ii) jetting a functional binder onto selected parts of said layer, wherein said functional binder infiltrates into pores in the powder bed and locally fuses particles of the powder bed in situ,

(iii) sequentially repeating said steps of applying a layer of powder on top and selectively jetting functional binder, multiple times, to provide a powder bed bonded at selected locations by printed functional binder.

The functional binder is applied from one or more inkjet heads. After the functional binder jetting process has been carried out to a desired extent, to part-form a 3D product, the top-most layer of the part-formed product acts as a substrate onto which is jetted a material, typically in a 2D pattern. Thus, a structure is printed onto said substrate. This structure may optionally be, and in many cases preferably is, a thin structure, for example one layer thick or two layers thick or a small number of layers thick, for example three layers thick or four layers thick or five layers thick, or one to five layers thick, or one to ten layers thick. It may optionally have a thickness, in the finished product, or 200 nanometers to 5 micrometers, or 300 nanometers to 3 micrometers, or 400 nanometers to 2 micrometers, or 500 nanometers to 1 micrometer. Typically, where a structure is formed of more than one layer, all or most of these layers may be the same. Nevertheless, it is possible that they may be different, such that there is some three-dimensional variation in the structure.

The structure is formed by jetting from one or more inkjet heads.

After the structure has been laid down, on the part-formed 3D product, the remainder of the 3D product is formed by continuing the functional binder jetting process on top of and optionally around at least some of, or in many cases, all of, said structure, so that the end result is a 3D product into which a structure is at least partially, and, in many cases, is completely, embedded in said 3D product. Said resultant bound 3D product may then be removed from the surrounding unbound powder.

This further functional binder jetting stage, like the first stage of functional binder jetting, also entails jetting functional binder from one or more inkjet heads.

Where the structure is more than one layer thick, there are different ways in which the surrounding bulk material can be built up. One way is to print the surrounding bulk material after each layer of structure is printed. An alternative way is to print the several layers of structure and then the equivalent of several layers of bulk material. In other words, the build material around the structure can be filled in either layer by layer or all at once.

Exemplary embodiments of the present disclosure facilitate the preparation of a 3D printed part which contains a structure embedded therein. Such structures can provide useful functionality. Examples of such embedded structures are sensors, actuators, security features, electronic features or other smart functionality.

Conventionally, where useful or functional structures (e.g. sensors, strain gauges, circuitry etc) have been embedded in 3D printed parts, this has typically been achieved by manufacturing such structures separately by other means and integrating them into the printed parts. This has generally been done by forming wells during additive manufacturing, stopping the additive manufacturing, placing the functional structures within the wells, and then resuming the additive manufacturing. Aspects of the present disclosure differ from this stop-start approach and provide a more efficient, seamless, accurate and advantageous approach whereby embedded functionality (i.e. the embedding of structures which are useful or can have a particular function) is effected by the jetting process itself.

The first stage of functional binder jetting, subsequent jetting of ink to form the structure which will become the embedded structure, and further stage of functional binder jetting to cap off the structure and complete the part, can be carried out seamlessly. This is because these steps all use the same process of jetting material “ink” from inkjet heads; they merely differ in that different inkjet heads, using different inks, are used in different stages of the process. In the first stage, functional binder is applied as an “ink” from a first inkjet head (or a plurality of inks is applied from a first set of inkjet heads) onto a powder bed. The printing of the structure (which will in the finished part become the embedded structure) is achieved by the application of one or more different inks from one or more different inkjet heads. The subsequent functional binder jetting stage can use the same inkjet head(s) as the first functional binder jetting stage, though in some cases it may be desired that the composition is different, in which case different inkjet head(s) using different ink(s) can be used.

Within each stage, it is possible that a single head may be used or that multiple heads may be used. For example, where a structure is to have a uniform composition, a single head could be used to apply material which contributes said uniform composition. Alternatively, even where a structure is to have a uniform composition, this could be achieved by co-jetting different materials from different heads onto the same locations. Furthermore, where a structure is to have a variable composition, for example where an alloy is to be applied in a graded fashion, this can be achieved by co-jetting different materials from different heads onto the same locations in variable amounts.

The structure may optionally comprise more than one sub-structure, and each sub structure may have a different function, for example a structure may comprise conductive track sub-structures and sensor sub-structures. Conventionally, additive manufacturing generally produces monomaterial structures, i.e. materials which have the same composition throughout the structure of a 3D printed part, i.e. are homogenous. Exemplary embodiments of the present disclosure enable the production of parts with multimaterials with control at the Voxel level, i.e. which are not homogenous but rather have structures of one composition or more than one composition embedded within a matrix of different composition.

At least one exemplary embodiment of the present disclosure include a process which enables a high level of detail and accuracy. The jetting of functional binder onto powder bed and the jetting of ink to produce embedded structures can both be carried out very accurately, and the advantages of functional binder jetting in terms of strength and other properties without the need for post-processing to remove or replace sacrificial binder mean that a finished complex part can be made effectively.

In contrast to some known processes, at least one exemplary process of the present disclosure chemically binds components together during the binder jetting stage. The at least one process may be considered to be a type of cementation. This chemical bonding, from the start of the process, differs from known processes which merely physically locate, or physically adhere, components together and then which require sintering or other treatment after printing. Some known processes produce green products which subsequently require fusing. Embodiments of the present disclosure can be advantageous in using chemical reactivity to produce binder-jetted, finished materials.

Whilst there are many types of embedded structure which can be included in accordance with exemplary embodiments of the present disclosure, it may be more clearly understood by considering one example, namely a strain gauge.

A strain gauge may take the form of a conductive strip which is arranged in a zig zag pattern. Stretching the zig zag structure results in slightly elongating each of the parallel lines making up the zig zag, the overall effect being to lengthen and narrow the conductive strip by an extent which results in a measurable change in electrical resistance. Measurement of a change in electrical resistance or conductance therefore indicates the amount of strain on an object containing the strain gauge, so long as the strain is transferred to the strain gauge, for example by embedding the strain gauge within the object. Suitable materials for conductive strips of strain gauges, and the reasons behind their suitability, are well known. One of many suitable materials is Constantan, a copper-nickel alloy, typically consisting of 55% copper and 45% nickel. Constantan exhibits reasonably constant resistivity over a range of temperatures, high strain sensitivity and good stability.

A sensor such as a strain gauge is an example of a functional structure which may be embedded in a 3D printed part in accordance with exemplary embodiments of the present disclosure. The functional structure has a pattern, here a 2D zig zag pattern, which is particularly suitable for formation by jetting onto a part-formed 3D product. For example, a Constantan strain gauge may be formed by jetting a nickel-containing ink (for example containing nickel acetyl acetonate) and concurrently jetting a copper-containing ink (for example containing copper formate).

Thus, a strain gauge or other functional structure may in effect be printed into a substrate. In the case of strain gauges, or other structures which function by virtue of their electrical properties, the substrate and other parts of the product may be of insulating material, for example ceramic material. Said ceramic material may itself by formed by the binder jetting of a suitable functional binder onto particles susceptible of being bound by said functional binder, for example onto a powder bed layer. The functional structure may be embedded, for example sandwiched within layers of insulating material.

Typically, the embedded functional structure may be a 2D printed pattern.

Another example of a suitable embedded functional structure is a printed platinum wire.

The functional binder jetting stages within exemplary methods of the present disclosure allow the production of end products which are functional products rather than prototypes. The functional binder is non-sacrificial: it contributes to the functional properties of the end product, e.g. properties of strength, rigidity, temperature-dependent behavior, stability, inertness, corrosion-resistance, conducting, insulating or electronic properties, so that the end product may be suitable for use as a product, part or component in for example the automotive, aerospace, military, marine, financial or medical device industries. Such products, parts or components may for example be components of vehicles or devices adapted to be used in or on the body.

The functional binder interacts with the surfaces of the powder bed particles so as to bind them together. The binder may do this directly or indirectly; in the latter case the binder may react during the jetting and/or deposition process to produce a more reactive species which then reacts with, and binds to, the surfaces of the powder bed particles.

The particles with which the functional binder interacts or reacts may be elemental powder particles, in which case the binder interacts with the surfaces of the elemental powder particles to bind them together. Alternatively or additionally the particles with which the functional binder interacts or reacts may be one or more compound, said compound itself reacting to form an elemental material. Thus it may be that, as well as the binder being a functional binder (i.e. a binder containing one or more compound that reacts to form an elemental material, as well as optionally containing elemental particles), the powder with which the functional binder reacts also contains one or more compound that reacts to form an elemental material, as well as optionally containing elemental particles. In other words, the functional binder contains a reactive component (i.e. a precursor of, or a compound of) an elemental material, and the particles with which said functional binder reacts may optionally contain a reactive component (i.e. a precursor of, or a compound of) an elemental material.

The binder may for example be a metallic binder, a ceramic binder or a polymeric binder, or may be a mixture, e.g. a mixture of a metallic binder and a ceramic binder, or different metallic binders. The binder may bind together the powder bed particles with elemental metal or may result in a part of the end product which comprises a metallic or non-metallic compound or component. Thus the binder may result in the end product containing a metal, e.g. copper, nickel, titanium, aluminum or cobalt, amongst others, or an oxide and/or nitride and/or carbide, amongst others, of aluminum, silicon, beryllium, cerium, zirconium, or other metals or non-metals.

Where the binder is a metallic binder, we term the functional binder jetting stages of the method “reactive metal jet fusion printing” (RMJF printing). In aspects of the present disclosure, the binder used is a functional (e.g. metallic) binder; the binder infiltrates into the voids between the powder-bed particles in situ, and the particles are fused in situ by application of the binder. The latter is due to the reaction with the functional binder and may also be facilitated by carrying out the process on powder particles at a higher temperature than is conventional (conventionally, in binder jetting methods, powder beds are not heated).

Without wishing to be bound by theory, chemical and physical processes are involved in forming the build material. The binder formulation may undergo a chemical transformation to for example result in a metal which physically fuses with the surrounding powder particles. The physical process may involve adsorption, diffusion and/or melting depending on the temperature.

The functional (e.g. metallic) binder contrasts with organic adhesive binders which have commonly been used prior to the method described in WO 2019/025801. At least one exemplary embodiment of the present disclosure allows the ink to be used as a means of incorporating metal or ceramic into the structure. The metal or ceramic remains in the end product even if a post-processing step of higher temperature sintering is carried out. This contrasts with, and brings advantages with respect to, the known use of sacrificial binders.

It should also be noted that exemplary embodiments of the present disclosure relate to the preparation of functional components or parts rather than mere prototypes. Binder jetting has been used in rapid prototyping: it enables 3D models to be produced easily. Such 3D models are not functional - their purpose generally relates to their appearance.

The infiltration of the binder into the voids between the powder-bed particles in situ differs from the conventional application of a binder which merely adhesively secures the powder bed layers. In the latter, significant porosity remains and this can lead to shrinkage or may require an infiltration procedure to be carried out in a post-processing step. In exemplary embodiments of the present disclosure, the in situ infiltration results in a simpler process and enables reliable manufacturing of structures whilst addressing shrinkage issues.

Optionally, the extent of infiltration may be such that the residual porosity by volume of the product prepared by the method of the first aspect, before post-processing, may be no greater than 30%, or no greater than 20%, or no greater than 10%, or no greater than 5%, or no greater than 1%. In comparison, the achievable density in a conventional powder bed is of the order of 60% due to constraints on packing densities, so that conventional residual porosities are of the order of 40%. An extensive level of infiltration may be achieved by the metal binder conformally coating the particles of the powder bed at a surface level. The binders fill, or partially fill, the interstices between the powder bed particles. The binders may contain molecular components which enable surface-driven reactions to bring about chemical fusing, in contrast to the binding provided by conventional binder jet printing.

The porosity may be measured by computed tomography (CT), e.g. according to the method described in Mattana et al, Iberoamerican Journal of Applied Computing, 2014, V. 4, N. 1, pp 18-28 (ISSN 2237-4523). The //7s/¾/fusing (e.g. joining, aggregation or bonding) of the particles with the metal of the binder brings further advantages compared to the use of a sacrificial adhesive binder; in particular the green strength of the material is enhanced, and composite and a wider range of tailored structures can be prepared.

Optionally one or more further step of post-processing may be carried out. In particular, the product may be heat-treated to consolidate and further strengthen, e.g. fuse, the structure. This may be done either after the application of each layer or after the entire structure has been built. The heat treatment step may be carried out at a temperature suitable for the material being used. For example, in some cases, it is beneficial to carry out a heat treatment step at a temperature towards, but not exceeding, the melting point of the material, e.g. steel 1100-1300, aluminum alloys 590-620, copper 750-1000, brass 850-950, bronze 740-780° C. It should be noted that this is a heat treatment step in contrast to the chemical process which occurs on application of the binder to the powder bed particles.

Thus the functional binder jetting stages of the present method facilitates the preparation of dense, optionally substantially fully dense, functional, 3D printed parts and in particular is a step forward with regard to metal additive manufacturing and ceramic additive manufacturing. Hitherto, only the powder bed fusion (PBF) technologies, such as selective laser melting (SLM), and more recently electron beam melting (EBM), have made significant inroads into the functional metal part market. These fusion based technologies, although impressive, have a number of problems, some related to the sub-optimal microstructure and others to scalability. The scalability has led to a limit on the size of objects that can be produced, lengthy manufacturing times, relatively high costs, problems with residual stress, and increasing difficulties with production as the size of the part increases. These problems have restricted SLM and EBM technologies to smaller, high added value-parts, and it is difficult to see how the technology can be scaled while controlling or reducing costs.

Exemplary embodiments of the present disclosure in effect combine the flexibility and agility of the laser powder melting techniques with the low cost of older powder bed print technologies, and furthermore provides new and advantageous ways of manufacturing printed parts with embedded structures.

Exemplary embodiments of the present disclosure benefit from some advantages of the binder jetting process compared to the powder bed fusion processes such as SLM and EBM (including: no support structures being required during the forming process, much higher layup speeds, ease of scaling and lack of internal stresses). At the same time exemplary embodiments of the present disclosure address an Achilles' heel of known binder jet technology in that it infiltrates the pores with metal or ceramic binder which makes the products suitable for use as functional components, and avoids using weak binders which can lead to the parts sagging during post processing.

The functional binder of various embodiments of the present disclosure is a material which may be applied by a jetting process to result in a metal, alloy or compound bound to the surfaces of the powder particles in the powder bed.

As discussed above, the binder is a functional binder, and may for example be a metallic binder or a ceramic binder. The binder may be in the form of a compound, salt or reagent, and may be in a carrier medium (e.g. a solvent), and the formulation may also comprise other components e.g. co-reagents (which may for example facilitate the conversion of compounds to elemental metals), other particles, and rheological agents to facilitate jetting, amongst other components. The binder may comprise a molecular precursor of a metal or alloy, for example an organometallic material. The organometallic material may be a compound or complex which can react in situ to result in a metal or alloy bound to the surface. The material may be referred to as a reactive organometallic ink because it is printed onto the substrate (powder bed) and reacts with the particulate material in the exposed powder bed layer.

Thus, whilst exemplary embodiments of the present disclosure are applicable to a range of functional binders, one important class is metallic binders. Metallic functional binder inks may contain reactive metal compounds, for example metal halides or metal salts, and amongst the most useful of reactive metal compounds are organometallics. Reactive organometallic (ROM) material undergoes reaction to lose ligands and change to elemental metal and bind to the particles.

Optionally the binder composition may comprise, in addition to a component which reacts at the molecular level (e.g. ROM), nanoparticles e.g. metal or ceramic nanoparticles. Optionally it may further comprise microparticles, e.g. metal or ceramic microparticles.

The metallic or ceramic binders (or inks) are capable of chemically fusing metal powders through a chemical transformation or conversion. During this process a metal adlayer or ceramic adlayer joins the particles and any filler particles. This is analogous to joining parts using a molten solder.

Optionally the metal or ceramic composition used in one or more embodiments of the present disclosure may have a size-distribution ranging from the molecular to nanoparticle through to the microparticle size or any mixture thereof. The purpose of having a range of different particle sizes is to achieve extensively or fully densified microstructures. Thus, while reactive materials e.g. organometallic (ROM) materials result in conformal coating of the particles (e.g. the powder bed particles) at the surface level, nano- and/or micro-particles fill the bulk of the voids or interstices. Therefore, optionally, the functional binder may comprise at least two components: a reactive material and a nanoparticulate and/or microparticulate material. Optionally the binder may comprise at least three components: a reactive material; a nanoparticulate material and a microparticulate material. Thus the skilled person will understand that a spectrum of particle sizes should be used in the binder (which may for example range from molecular materials to nanoparticulate materials to microparticulate materials), to enable the space and interstices between the particles to be effectively filled. The most effective distribution of particle sizes to be used is preordained by the nature of the components making up the particulate material (e.g. the powder bed particulate material). The present inventors have recognised that, for any particular desired final material, a suitable matrix for the particulate material can be chosen, and that this then predetermines the distribution of particle sizes of the “ink” which will be appropriate to produce a fully-filled, fully-functional material.

The functional binder or “ink” can therefore be considered to be a hierarchical ink, i.e. one that contains a range of materials of different sizes and natures, for example ranging from reactive organometallic materials to particulate materials, optionally particulate materials having a size distribution.

By nanoparticulate is meant that the particle size is on average within the ranges 1 to 100 nm, or 5 to 100 nm, or 1 to 50 nm, or 1 to 20 nm, or 1 to 10 nm, or 2 to 8 nm, or 3 to 7 nm, or about 5 nm).

By microparticulate is meant that the particle size in the ink is on average within the ranges 0.1 to 10 microns, or 0.1 to 5 microns, or 1 to 5 microns, or 1 to 3 microns.

Thus it may be that the binder composition may comprise three components which, along with the particles (e.g. the powder bed particles) intended to be bound by said binder, form the build material: a functional binder fraction, a nanoparticulate fraction and a microparticulate fraction. It may be that the functional binder fraction forms 0.1-10%, e.g. 0.5-8%, e.g. 0.7-2%, e.g. 0.8-1.2%, e.g about 1%, of the volume of the product. It may be that the nanoparticulate fraction and the microparticulate fraction together form 10-50%, e.g. 20-45%, e.g. 30-40%, e.g. 35-40% of the volume of the product. It may be that the ratio of nanoparticulate to microparticulate fraction in the product, by volume, is between 10:1 and 1:10, e.g. between 5:1 and 1:5, e.g. between 2:1 and 1:2, e.g. between 10:1 and 1:1, e.g. between 5:1 and 2:1, e.g. between 1:1 and 10:1, e.g. between 2:1 and 5:1. The skillset of those working in 3D printing has generally not included detailed chemistry expertise. The inventive approach described herein arises in part from an understanding of how to use chemical components to form embedded structures within multimaterial parts.

From further aspects the present disclosure provide functional binder compositions used in exemplary methods of the present disclosure.

The inks infiltrate the porosity (typically about 40% porosity) in the particulate material (e.g. the powder bed lay-up). The infiltrated material may optionally comprise up to 20% by volume of reactive binder (e.g. ROM) with the balance being comprised of particles, other components and carrier. Together these components act as an infiltrating metallic or ceramic binder to hold the 3D part in a green state until it can be subsequently consolidated by heat treatment. By filling the powder lay-up with metal or ceramic binder the final porosity, distortion and shrinkage of the finished part are reduced.

Metals printed in accordance with exemplary embodiments of the present disclosure include copper, nickel, titanium, aluminum and cobalt and alloys thereof. Ceramics printed in accordance with exemplary embodiments of the present disclosure include alumina and other materials including oxides and/or nitrides and/or carbides, amongst others, of aluminum, silicon, beryllium, cerium, zirconium, or other metals or non-metals. Cermets and oxide dispersion strengthened materials may also be produced. Exemplary embodiments of the present disclosure allow the production of materials which have active material parts, e.g. shape memory alloys, piezoelectric materials, etc.

In the case of metallic binders, optionally aspects of the present disclosure utilize volatile metal precursor (reactive organometallic (ROM)) compounds), developed for chemical vapor deposition processes, as the basis for ink formulations.

Aside from ROMs, other materials may be used including for example salts, halides, alkyls, alkylamides, silylamides, organophosphorous compounds, organosulphurous compounds, organohalides, ketones and aldehydes, amongst others. The inks may incorporate certain concentrations of the ROM component (e.g. about 5-50%, e.g. 10-40%, e.g. 20-30%, w/w) combined with certain loadings of metal micro- and nano-particles (e.g. about 10-60%, e.g. 20-50%, e.g. 30-40%, w/w). The melting temperature of very small nanoparticles is typically suppressed compared with the bulk, because the relief of the very high surface energy: volume ratio provides the thermodynamic driving force for melting or sintering. Optionally further components may be present, for example to control the reactivity of metal nanoparticles towards unwanted reactions (e.g. oxidation) before they can be incorporated into the 3D metal part. The use of pre-treatments can “cap” or encapsulate the nanoparticles in a protective layer to stop oxidation. Optionally ionic surfactants (e.g. Brij™ or Tween™) may be used to deliver metallic fillers into the porosity left by the feedstock powder. For larger micron-scale filler metal particles encapsulation is generally not necessary; however optionally the surface passivation layers on these particles may be reduced via a range of reducing pre treatments. Optionally encapsulation may be used to reduce the extent of unwanted native oxide into the RMJF 3D parts. Optionally viscosity modifiers and surfactants may be used to inhibit particle agglomeration in order to suspend the metal particulates in the ROM solutions.

Some examples of materials to which some embodiments the present disclosure is applicable include aluminum and its alloys, shape memory alloys, oxide strengthened alloys, tungsten and tantalum alloys, steels, magnesium materials, ceramics and glasses. For example, magnesium can be made fireproof or corrosion-resistant by application of a surface matrix surrounding the powder.

Any suitable material may be used as the particles (e.g. powder bed particles) intended to be bound by the functional binder, including those which are conventional used in powder beds. These include metals and ceramics, or mixtures thereof.

From further aspects the present disclosure provide 3D printed products obtained or obtainable by exemplary methods of the present disclosure. These are distinguishable from products made by other methods because of their properties, for example the embedded nature of functional structures. Exemplary embodiments of the present disclosure allow the preparation of products which have properties suitable for their function.

Because the binder used in exemplary embodiments of the present disclosure is not a sacrificial binder and becomes part of the build material, the resultant product can exhibit improved properties structurally (e.g. strength or fatigue resistance), in terms of conductivity (electrically or thermally), or in other ways. Without wishing to be bound by theory, exemplary embodiments of the present disclosure ameliorate the flaws in the product due to cracks and porosity thereby improving the mechanical properties.

It may be that a product, component, or part made in accordance with exemplary embodiments of the present disclosure is an automotive part, an aerospace component, an engineering component, a marine plant component, a component used in a military or weaponry application, a structural component, a medical device component, an implant or component thereof, or a prosthesis or component thereof.

It may be that the product has a porosity (of the embedded functional part) of less than 10%, or less than 5%, or less than 1% of the bulk volume.

It may be that the product has a porosity (overall) of less than 10%, or less than 5%, or less than 1% of the bulk volume.

The inkjet binder printer used may be based on TTPs “Vista” technology print heads.

The binder jet printer is capable of printing metallic functional binders for multiple materials and layering metal powder feed stocks.

Optionally the binder printing system incorporates print heads that are capable of jetting micron-sized particles. This binder printing system enables flexibility in the use of a range of binder inks and produces a print system that is capable of building complex 3D components beyond what is currently feasible using known procedures.

From a further aspect of the present disclosure provides apparatus for carrying out one or more methods of the present disclosure. The skilled person will understand that the different components of the binder may play different roles.

The nanoparticulate material may allow the sintering temperature to be reduced and plays a role in reducing porosity. It becomes part of the build material (i.e. is non-sacrificial).

The microparticulate material also plays a role in reducing porosity, at a different level. It becomes part of the build material (i.e. is non-sacrificial).

The ROM or other molecular material may help carry the particulate material to facilitate jetting, may bind the powder bed together, and converts to a material (e.g. metal or ceramic) which becomes part of the build material (i.e. is non-sacrificial).

Thus the conformal coating and reaction facilitated by the ROM or other molecular material, in combination with the further space-filling provided by the other components, and the sintering to produce a fully-filled, fully-functional, material, bring about considerable advantages compared to known disclosures. Waste and burn-off of materials are avoided, and the product has improved properties.

Alloys and other composite materials may be made by for example using a component (e.g. the microparticulate component or alternatively/additionally one of the other components) which is different to the powder bed material.

Further functionalisation may be brought about by for example using functionalised nanoparticles (or functionalised other components) to embed other properties into the final material.

Suitable compositions (“inks”) for the material which is jetted to form the structure which will be embedded can include metallic materials, organometallic materials, ceramic materials and polymer materials. “Polymer materials” can optionally include materials which polymerise in situ. Exemplary embodiments of the present disclosure can be advantageous in allowing a range of types of embedded structure to be incorporated. Some of the suitable compositions (“inks”) for the material which is jetted to form the structure which will be embedded can include the same compositions (“inks”) which are suitable for use as functional binders. In particular, inks containing organometallic materials, including for example hierarchical inks with a distribution of particles sizes, are highly effective. Without wishing to be bound by theory, we believe that the use of organometallic materials, e.g. reactive organometallic materials, leads to surface-driven chemistry when the structure is printed which results in effective adherence on the substrate and enables material interface control at the molecular level. At least one exemplary embodiment of the present disclosure allows dissimilar materials to be compatible with each other and to bind together at their surfaces. The 2D structure can thus be effectively printed on the part-made 3D product.

Furthermore, in some cases the functional binder jetting process can be used to form not only the parts of the 3D product which surround the embedded structure but also the embedded structure itself. For example, after the first functional binder jetting stage, which is designed to part-form the main matrix of the product, the structure which is to be embedded may be printed by using a different functional binder-powder layer combination: a functional binder may be jetted in a suitable pattern on a powder bed layer to form the embedded structure. Subsequently the patterned structure is covered by a further binder jetting stage.

At least one exemplary embodiment of the present disclosure will now be described in further non-limiting detail and with reference to the drawings in which:

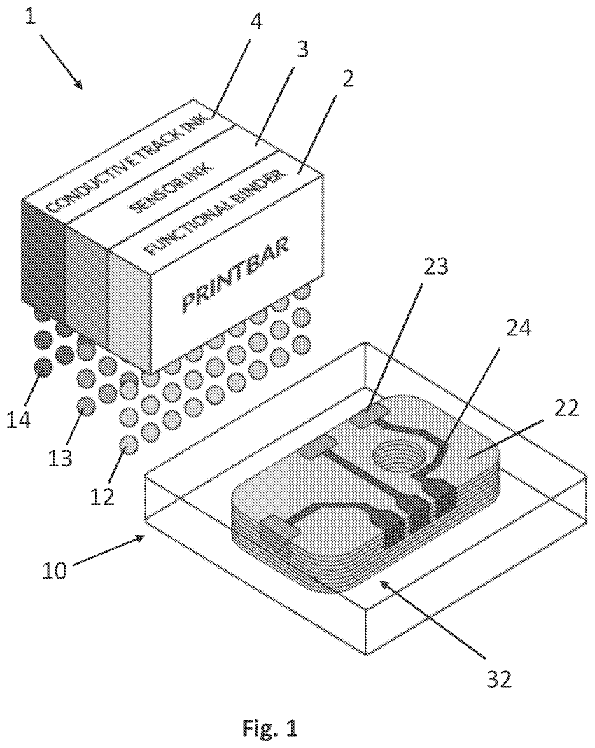

FIG. 1 is a schematic representation of apparatus used and components prepared in one embodiment of a method of 3D printing in accordance with at least one exemplary embodiment of the present disclosure; and

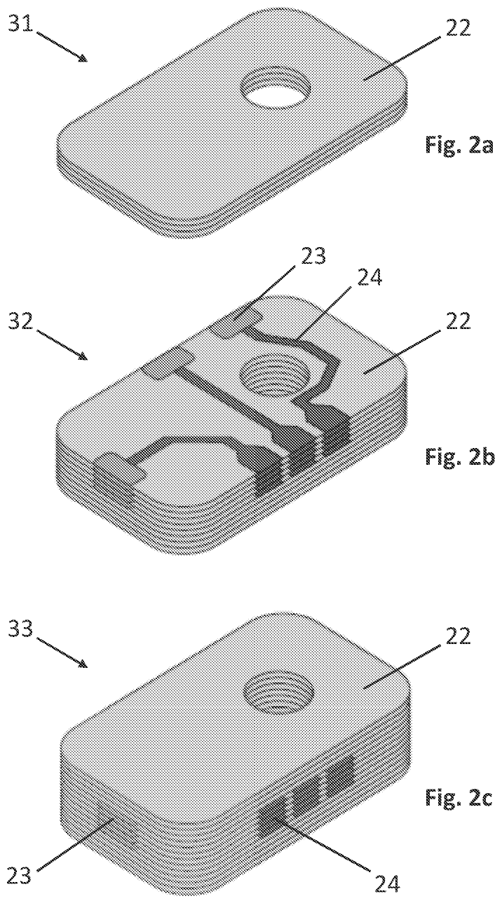

FIGS. 2a, 2b and 2c are schematic representations of incomplete, part-finished and finished components prepared in accordance with at least one exemplary embodiment of the present disclosure.

It will be understood that the illustrated embodiment shows merely one of many possible examples of how aspects of the present disclosure may be realized. With reference to FIG. 1, a 3D printer 1 is used to print a 3D printed product (a part-finished 3D printed product 32 is shown) in a powder bed apparatus 10. Said part-finished 3D printed product 32 is also shown in FIG. 2b, and the same component during earlier and later stages of the 3D printing process is shown in FIGS. 2a and 2c respectively.

The 3D printer 1 jets various “inks” from different print heads.

One type of ink is functional binder ink 12 from a printbar component 2. This is jetted onto a powder bed layer (not shown) and infiltrates into pores in the powder bed and locally fuses particles of the powder bed in situ to form build material 22 which forms the bulk of the 3D printed product. FIG. 2a shows the product 31 at a preliminary stage when functional binder 12 has been printed onto a layer of powder and this process has been repeated several times to form several layers composed exclusively of build material 22.

Other ink is used to form the embedded structure, and in the illustrated embodiment there are two types of such ink: sensor ink 13 from printbar component 3, to form sensors 23; and conductive track ink 14 from printbar component 4, to form conductive tracks 24. FIG. 2b shows the product at an intermediate stage when several layers of said structure (which comprises two sub-structures: conductive tracks 24 and sensors 23) have been printed. Build material 22 surrounds the embedded structure.

After the desired amount of structure (formed of sensors 23 and conductive tracks 24) has been printed, further build material 22 may be formed in layers on top of said structure to form product 33 (FIG. 2c).

For ease of understanding the process, separate layers are shown in products 31, 32 and 33, but in practice these are not visible in the products produced.

While the present disclosure has been particularly shown and described with reference to specific embodiments, it should be understood by those skilled in the art that various changes in form and detail may be made therein without departing from the spirit and scope of the invention as defined by the appended claims. Further, while the present disclosure is presented in terms of exemplary embodiments, it should be appreciated that individual aspects of the disclosure can be separately claimed. The scope of the invention is thus indicated by the appended claims, and all changes within the meaning and range of equivalency of the claims are therefore intended to be embraced.

What is claimed is:

1. A method of 3D printing in which a 3D product is built up layer by layer by jetting from print heads, comprising:
(a) forming part of a 3D product by a functional binder jetting process;
(b) jetting one or more material in a 2D pattern to form a structure on said part;
(c) completing the formation of the 3D product by continuing the functional binder jetting process, so that said structure becomes embedded in said product.

2. The method as claimed in claim 1 wherein said functional binder jetting comprises:
(i) providing a layer of a powder bed;
(ii) jetting a functional binder onto selected parts of said layer, wherein said functional binder infiltrates into pores in the powder bed and locally fuses particles of the powder bed in situ, (iii) sequentially repeating applying a layer of powder on top and selectively jetting functional binder, multiple times, to provide a powder bed bonded at selected locations by printed functional binder

3. The method as claimed in claim 1, wherein the bulk of said 3D product, formed by said functional binder jetting, is a metal, alloy or ceramic or mixture of the same.

4. The method as claimed in claim 1, wherein said embedded structure is a metal, alloy or ceramic structure.

5. The method as claimed in claim 1, wherein said embedded structure is a polymer structure.

6. The method as claimed in claim 1, wherein said embedded functional structure is selected from one or more of sensors, actuators, security features, electronic features or smart functionality.

7. The method as claimed in claim 6 wherein said embedded functional structure is a strain gauge.

8. The method as claimed in claim 1, wherein the functional binder comprises a metallic binder.

9. The method as claimed in claim 8, wherein the metallic binder is an organometallic material.

10. The method as claimed in claim 1, wherein the functional binder comprises a ceramic binder.

11. The method as claimed in claim 1, wherein the functional binder further comprises metallic or ceramic nanoparticles with sizes within the range of 1 to 100 nm.

12. The method as claimed in claim 1, wherein the functional binder further comprises metallic or ceramic microparticles with sizes within the range of 0.1 to 10 microns.

13. The method as claimed in claim 1, wherein said material jetted in a 2D pattern to form said structure comprises a metallic material.

14. The method as claimed in claim 1, wherein said material jetted in a 2D pattern to form said structure comprises an organometallic material.

15. The method as claimed in claim 14, wherein the organometallic material is a copper metal precursor and/or isocyanide ligands, or is a nickel metal precursor, or is a titanium metal precursor.

16. The method as claimed in claim 1, wherein said material jetted in a 2D pattern to form said structure comprises an ceramic material.

17. The method as claimed in claim 13, wherein said material jetted in a 2D pattern to form said structure further comprises metallic or ceramic nanoparticles with sizes within the range of 1 to 100 nm.

18. The method as claimed in claim 13, wherein said material jetted in a 2D pattern to form said structure further comprises metallic or ceramic microparticles with sizes within the range of 0.1 to 10 microns.

19. The method as claimed in claim 1, wherein said material jetted in a 2D pattern to form said structure comprises a polymer material.

20. The method as claimed in claim 1, further comprising a heat treatment to further fuse the 3D product.

21. A 3D printed product obtainable by the method of claim 1.

22. A 3D printed product formed of fused particles of metal and/or ceramic containing an embedded structure.

23. (canceled)

24. (canceled)

25. The method as claimed in claim 8,
wherein the organometallic material is a copper metal precursor and/or isocyanide ligands, or is a nickel metal precursor, or is a titanium metal precursor.
